(19) United States
(12) Patent Application Publication
(10) Pub. No.: US 2025/0057866 A1
(43) Pub. Date: Feb. 20, 2025

(54) A SYNERGISTIC PHARMACEUTICAL COMPOSITION AND ITS ANTIVIRAL USE

(71) Applicant: Guangzhou Anobri Pharmaceutical Co. Ltd., Guangdong (CN)

(72) Inventors: Cai Gu Huang, Guangdong (CN); Hong Da Zhuang, Guangdong (CN); Hui Sun, Guangdong (CN)

(21) Appl. No.: 18/723,921

(22) PCT Filed: Apr. 1, 2022
(86) PCT No.: PCT/CN2022/084724

§ 371 Date: Jun. 25, 2024

(30) Foreign Application Priority Data
Feb. 14, 2022 (CN) 202210134422.2

(51) Int. Cl. A61K 31/7068; A61K 31/4025; A61P 31/14

(52) U.S. Cl. CPC A61K 31/7068; A61K 31/4025; A61P 31/14

(57) ABSTRACT

A pharmaceutical composition containing a deuterated cyano compound, isomer, prodrug, solvate, or pharmaceutically acceptable salt thereof, and a deuterated cytidine derivative, isomer, prodrug, solvate, or pharmaceutically acceptable salt thereof, and its use as an antiviral.

### CROSS-REFERENCE TO RELATED APPLICATIONS

This application is a national stage filling under 35 U.S.C § 371 of international application number PCT/CN2022/084724, filed Apr. 1, 2022, which claims priority to Chinese patent application No. 202210134422.2, filed Jan. 20, 2022. The contents of these applications are incorporated herein by reference in their entirety.

### BACKGROUND OF THE INVENTION

SARS virus belongs to the order of nested viruses, Coronaviridae and Coronaviruses, and is a subset B coronavirus of β genus. The virions are enveloped and surrounded by coronal filaments, distributed in the cytoplasm, and are round in shape. The diameter of the virus ranges from 80 nm to 120 nm. SARS is an infectious disease with rapid onset, rapid spread, and high mortality. Most patients who get infected are in direct or indirect contact with infected patients, or live in endemic areas.

MERS-COV is a subset the C Coronavirus of β genus, which is named Middle East Respiratory Syndrome Coronavirus (MERS-COV). MERS-COV was first discovered in Saudi Arabia in September 2012. MERS-COV was named a “SARS-like virus” because its clinical symptoms were similar to SARS, and it became the sixth known human Coronavirus and the third species isolated in the past 10 years.

The novel coronavirus SARS-COV-2 is a coronavirus strain that previously had not been found in humans. It was first discovered and reported in 2019. It is still prevalent in many countries worldwide and is not well controlled in many countries and regions.

The common signs of a coronavirus infection include respiratory symptoms, fever, cough, shortness of breath, and dyspnea. In more severe cases, infection can lead to pneumonia, severe acute respiratory syndrome, renal failure, and even death, and currently there is no specific treatment for diseases caused by novel coronaviruses.

A novel deuterated cyano compound was developed by Shanghai Anovent Pharmaceutical Co., Ltd. as a small molecule 3CL protease inhibitor The structure is as follows:

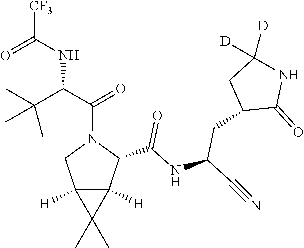 [SMILES: [2H]C1([2H])C[C@@H](C[C@@H](C#N)NC(=O)[C@@H]2[C@@H]3[C@H](CN2C(=O)[C@@H](NC(=O)C(F)(F)F)C(C)(C)C)C3(C)C)C(=O)N1]

The compound has excellent anti-SARS-COV-2 activity and can effectively inhibit virus replication in vitro. On the basis of comparable antimicrobial activity, the compound achieves better pharmacokinetic properties than other oral anti-SARS-COV-2 virus drugs. Shanghai Anovent Pharmaceutical Co., Ltd. has applied for a patent (Application No. CN202111234708X) for the novel deuterated cyano compound.

A deuterated cytidine derivative is another small molecule neocytidine antiviral oral drug independently developed by Shanghai Anovent Pharmaceutical Co., Ltd. This drug is a SARS-CoV-2 polymerase inhibitor, which has strong anti-SARS-COV-2 activity and can effectively inhibit virus replication in vitro. The chemical name of the deuterated cytidine compound is ((2R, 3S, 4R, 5R)-3,4-dihydroxy-5-(4-(hydroxylamino)-2-oxopyrimidin-1 (2H)-yl) tetrahydrofuran-2-yl) deuterated methyl isobutyl ester, and a patent has been applied for (Application No. CN2021110245289). The structure is:

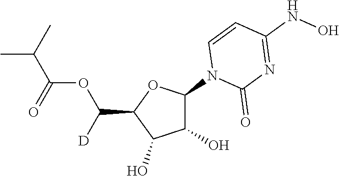 [SMILES: [2H]C(OC(=O)C(C)C)[C@H]1O[C@@H](n2ccc(NO)nc2=O)[C@H](O)[C@@H]1O]

Although the deuterated cyano compounds and the deuterated cytidine derivative have good antiviral activity in vitro, their safety and drug resistance needs to be improved. Based on this, there is an urgent need to develop effective pharmaceutical compositions to improve the antiviral activity, drug resistance, and safety of antiviral drugs. The pharmaceutical compositions disclosed herein address these problems.

### SUMMARY OF THE INVENTION

The invention provides pharmaceutical compositions comprising a deuterated cyano compound or isomers, prodrug, solvate, or pharmaceutically acceptable salt thereof, and a deuterated cytidine derivatives or isomer, prodrug, solvate, or pharmaceutically acceptable salt thereof, and their use as an antiviral agent.

The deuterated cyano compound and the deuterated cytidine derivative used in the pharmaceutical compositions and methods of the invention are a small molecule 3CL protease inhibitor and a neocytidine antiviral oral drug, each developed by Shanghai Anovent Pharmaceutical Co., Ltd., which exhibit excellent antiviral activity.

The pharmaceutical composition of the present invention has the following advantages and beneficial effects:

The antiviral effect of the composition of the invention is significantly superior to that of either drug individually and exhibits a significant synergistic effect that reduces cytotoxicity.

The deuterated cyano compound targets viral proteases, while the deuterated cytidine derivatives targets viral RNA polymerase. Combining drugs that interact with different targets reduces the problem of drug resistance and thus improves drug safety.

### BRIEF DESCRIPTION OF DRAWINGS

FIG. 1 illustrates the effect of a pharmaceutical composition on cytotoxicity.

FIG. 2 illustrates the antiviral activity of a pharmaceutical composition.

FIG. 3 illustrates the cellular antiviral activity of the pharmaceutical composition compared to that of each drug individually.

### DETAILED DESCRIPTION OF THE INVENTION

The invention is directed to pharmaceutical compositions comprising a deuterated cyano compound, isomer, prodrug, solvate, or pharmaceutically acceptable salt thereof, and a deuterated cytidine derivative, isomer, prodrug, solvate, or pharmaceutically acceptable salt thereof, and uses of the composition as an antiviral.

In one aspect, the invention is directed to a pharmaceutical composition comprising a deuterated cyano compound, isomer, prodrug, solvate, or pharmaceutically acceptable salt thereof, and a deuterated cytidine derivative, isomer, prodrug, solvate, or pharmaceutically acceptable salt thereof.

The structure of the deuterated cyano compounds is:

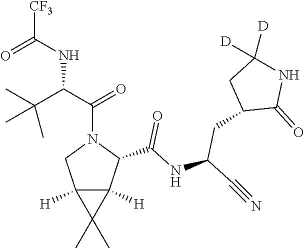 [SMILES: [2H]C1([2H])C[C@@H](C[C@@H](C#N)NC(=O)[C@@H]2[C@@H]3[C@H](CN2C(=O)[C@@H](NC(=O)C(F)(F)F)C(C)(C)C)C3(C)C)C(=O)N1]

The structure of the deuterated cytidine derivative is:

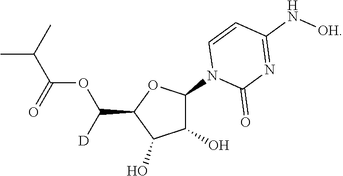 [SMILES: [2H]C(OC(=O)C(C)C)[C@H]1O[C@@H](n2ccc(NO)nc2=O)[C@H](O)[C@@H]1O]

Pharmaceutically acceptable salts include organic and inorganic salts of the respective compounds, which may be any salt well known to those skilled in the art. Preferred but non-limiting examples include hydrochloride salts, hydrobromide salts, sulfate salts, nitrate salts, acetate salts, adipate salts, aspartate salts, benzoate salts, and the like.

Further, the compounds may be administered in the form of a prodrug. The prodrug may comprise a covalently bonded molecule that releases the active parent drug when administered to a mammalian subject. The prodrug may be cleaved into the parent compound in vivo.

Solvates of the compounds refer to an association of one or more solvent molecules with the compound. Solvents that can form solvates include, but are not limited to, water, isopropanol, ethanol, methanol, dimethyl sulfoxide, and the like.

Isomers of the invention include stereoisomers and tautomers.

In addition, the compounds of the invention include crystalline and amorphous forms of the compounds, wherein crystals include single crystals, polycrystals, and co-crystals.

Preferably, the weight ratio of the deuterated cyano compound, isomer, prodrug, or pharmaceutically acceptable salt thereof, to the deuterated cytidine derivative, isomer, prodrug, solvate, or pharmaceutically acceptable salt thereof is about (100-300):(200-400). Preferably, their weight ratio is about 100:200, 100:300, 100:400, 200:300, 200:400, or 300:400. More preferably, their weight ratio is about 200:300.

Preferably, the pharmaceutical compositions include from about 100 mg to about 300 mg of the deuterated cyano compound and from about 200 mg to about 400 mg of the deuterated cytidine derivative. In one embodiment, the pharmaceutical compositions include about 200 mg of the deuterated cyano compound and about 200 mg to about 300 mg of the deuterated cytidine derivative. In one embodiment, the pharmaceutical compositions include about 200 mg of the deuterated cyano compound and about 300 mg of the deuterated cytidine derivative.

It will be appreciated that the deuterated cyano compound and the deuterated cyano compound may be administered simultaneously in the same or different pharmaceutical compositions, or may be administered sequentially, i.e., the deuterated cyano compounds and the deuterated cytidine derivatives may administered in individual dosing units or they can be combined together to form a single dosing unit. If administered independently, the interval between the administration of the first active agent and the second active agent should not lose the benefit of the synergistic efficacy that is achieved by the combination of active agents. In one embodiment, the deuterated cyano compound and the deuterated cytidine compound are administered as independent dosing units. In one embodiment, the deuterated cyano compound and the deuterated cytidine compound are combined into a single dosing unit for administration.

The pharmaceutical compositions can further include a pharmaceutically acceptable carrier consisting of a material that is considered safe and effective when administered to an individual and does not cause undesirable biological side effects or undesired interactions when administered. The term “carrier,” as used herein refers to all components present in a pharmaceutical composition except the active ingredient. As commonly used herein, carriers include, but are not limited to, modifiers, adhesives, lubricants, disintegrants, fillers, colorants, plasticizers, film-coating materials, organic solvents, solubilizers, flavoring agents, surfactants, and the like.

A filler, also known as a diluent, is generally necessary to increase the volume of a solid dosage form so as to provide a practical size for compression into a tablet or forming beads or particles. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starch, pregelatinized starch, silica, titanium oxide, magnesium alumina, and sugar powder.

Adhesives are used to impart adhesiveness to solid dosage forms, such as tablets, beads, or particles, thereby ensuring that the solid dosage form remains intact after the dosage form is formed. Suitable adhesive materials include, but are not limited to, starch, pregelatinized starch, gelatin, sugars (including sucrose, glucose, dextrose, lactose, and sorbitol), polyethylene glycols, waxes, natural gums, and synthetic gums.

Lubricants are used to facilitate the manufacture of tablets. Examples of suitable lubricants include, but are not limited to, magnesium stearate, calcium stearate, stearic acid, glyceryl behenate, polyethylene glycol, talc, and mineral oils.

Disintegrants are used to facilitate disintegration or decomposition of the dosage form after administration. Disintegrants include, but are not limited to, starch, sodium starch glycolate, sodium carboxymethyl starch, sodium carboxymethylcellulose, hydroxypropyl cellulose, pregelatinized starch, clay, cellulose, alginate, gum, and crosslinked polymers.

Stabilizers are used to inhibit or delay drug decomposition reactions, e.g., oxidation reactions.

The surfactant may be an anionic surfactant, a cationic surfactant, an amphoteric surfactant, or a nonionic surfactant. Suitable anionic surfactants include, but are not limited to, those containing carboxylate, sulfonate, and sulfate ions. Examples of anionic surfactants include, but are not limited to, sodium, potassium, and ammonium salts of long chain alkylsulfonic acids and alkylarylsulfonic acids, such as sodium dodecylbenzene sulfonate and sodium dialkylsulfosuccinate.

Examples of plasticizers include, but are not limited to, polyethylene glycol, propylene glycol, triacetin, dimethyl phthalate, diethyl phthalate, dibutyl phthalate, dibutyl sebacate, triethyl citrate, tributyl citrate, acetyl triethyl citrate, castor oil, and acetylated monoglycerides.

Organic solvents include, but are not limited to, alcohols, such as isopropanol, propylene glycol, polyethylene glycol, polypropylene glycol, glycerol, polyoxyvinyl alcohol, and the like.

The pharmaceutical compositions described herein may be formulated for regulating or controlling release of the active agents. Examples of controlled release dosage forms include, but are not limited to, extended release dosage forms, delayed release dosage forms, pulse release dosage forms, and combinations thereof.

Preferably, the compound or pharmaceutical composition containing the compound is administered orally or intravenously. Oral administration includes, but is not limited to, administering tablets and liquids.

In another aspect, the invention provides for the use of the deuterated cyano compound and the deuterated cytidine compound to prepare a medicament for the treatment or prophylaxis of a viral infection, which may be the novel coronavirus SARS-COV-2, SARS-coronavirus, MERS-coronavirus, eastern equine encephalitis virus, western equine encephalitis virus, Venezuelan equine encephalitis virus, chikungonian fever virus, orthomyxovirus or paramyxoviridae virus, RSV virus, influenza A and influenza B virus, or Ebola virus. Preferably, the virus is the novel coronavirus SARS-COV-2, SARS-coronavirus, or MERS-coronavirus. More preferably, the virus is the novel coronavirus SARS-COV-2.

The deuterated cyano compound and deuterated cytidine compound used in the pharmaceutical compositions and methods of the invention are a small molecule 3CL protease inhibitor and a neocytidine antiviral oral drugs developed by Shanghai Anovent Pharmaceutical Co., Ltd., which exhibit excellent antiviral activity.

The pharmaceutical compositions of the invention have the following advantages and beneficial effects:

The antiviral effect of the compositions of the invention are significantly superior to that of either drug individually, and exhibits a significant synergistic effect that reduces cytotoxicity.

The deuterated cyano compound targets viral proteases, while the deuterated cytidine derivatives target viral RNA polymerase. Combining drugs that interact with different targets reduces the problem of drug resistance and thus improves drug safety.

### EXAMPLES

The present invention will be described in detail with following specific embodiments.

The following detailed descriptions are exemplary and explanatory only and should not be considered as limiting.

In the following examples, all solvents and reagents used are commercially available and used as is, unless otherwise indicated.

### Example 1: Synthetic Route of the Deuterated Cyano Compound (Hereinafter Referred to as Compound 1)

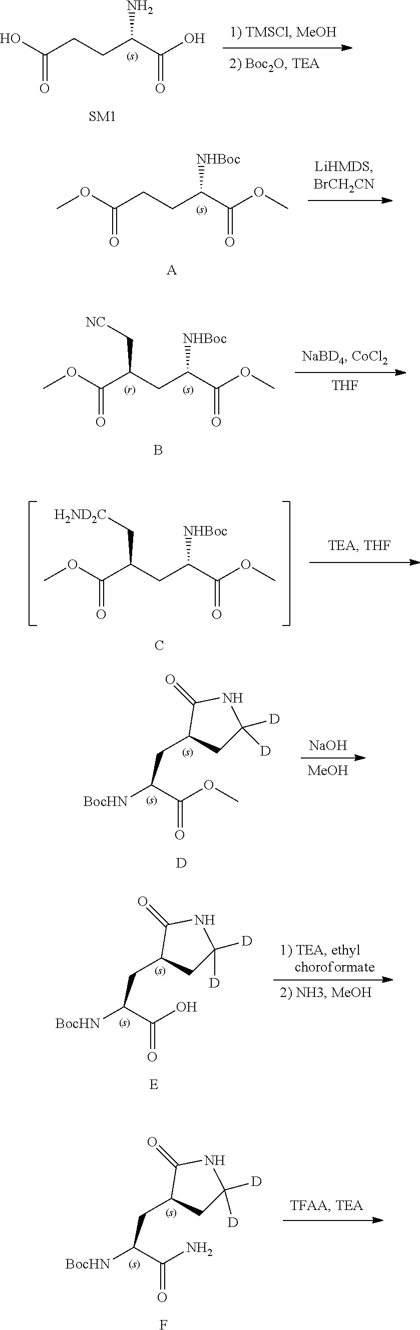

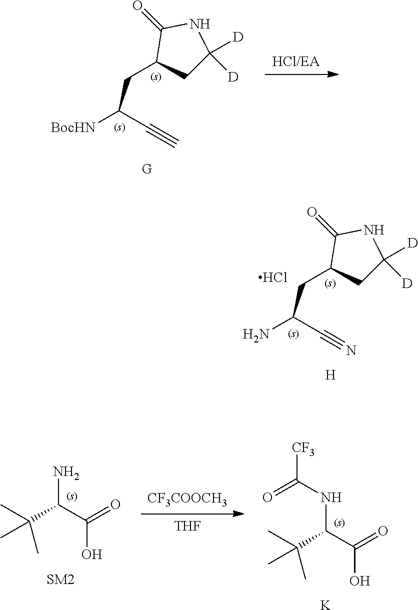

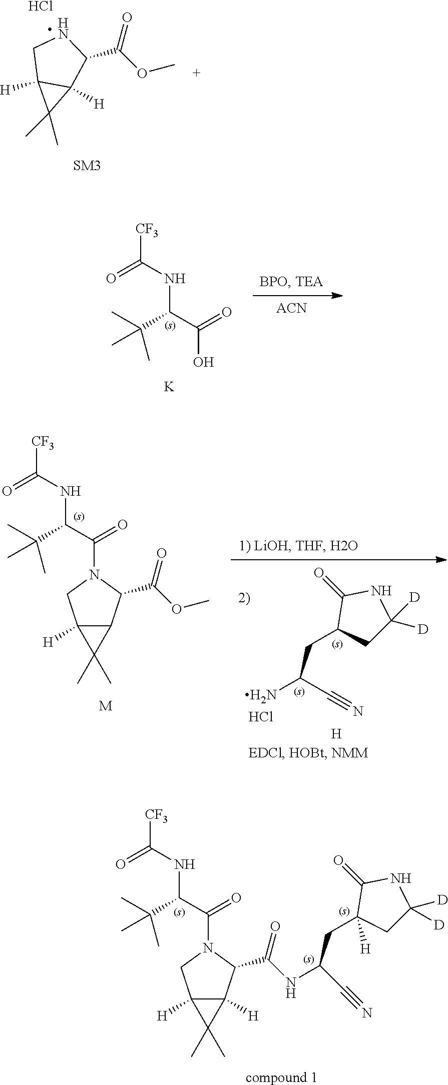

The preparation of intermediates H, K, M can be found in the previous application of the inventors, CN202111234708.X, the contents of which are expressly incorporated herein.

### Synthesis of the Deuterated Cyano Compounds (Hereinafter Referred to as Compound 1)

An aqueous solution of lithium hydroxide (1.5 g, 62.8 mmol, 15 mL) was added to a stirred solution of M (8 g, 21.1 mmol) in THF (80 mL) and MeOH (80 mL) at room temperature. The resulting solution was stirred at room temperature for 2 hours and then cooled to about 0 to about 10° C., acidified with 1N HCl, and extracted with ethyl acetate (EA). The aqueous layer was separated and extracted twice with EA. The organic layers were combined, dried with anhydrous magnesium sulfate, and concentrated to dryness to provide a crude product that was purified by column chromatography to obtain hydrolyzed M.

Hydrolyzed M obtained in the previous step and dimethylformamide (DMF) (100 mL) were combined in a three-necked flask and cooled to 0-10° C. 1-Ethyl-3-(3-dimethylaminopropyl) carbodiimide (EDCI) (4.9 g, 25.6 mmol), 1-hydroxybenzotriazole (HOBt) (3.4 g, 25.2 mmol), and N-methylmorpholine (NMM) (4.3 g, 42.5 mmol) were added sequentially and the resulting mixture stirred for 30 minutes at about 0 to about 10° C. Compound H (4.0 g, 21 mmol) was added in batches and the resulting mixture stirred at room temperature overnight. The reaction was monitored until shown to be complete by TLC. After adding water (80 mL), the suspension was extracted three times with EA. The organic phases were separated and combined; washed sequentially with 0.5 N HCl, 5% NaHCO₃ aqueous solution, and water; dried with anhydrous magnesium sulfate; and concentrated to provide a crude product that was purified by column chromatography to provide Compound 1 (8.4 g) as an off-white solid.

### Example 2: Route of Synthesis of Deuterated Cytidine Derivative (Hereinafter Referred to as Compound 2)

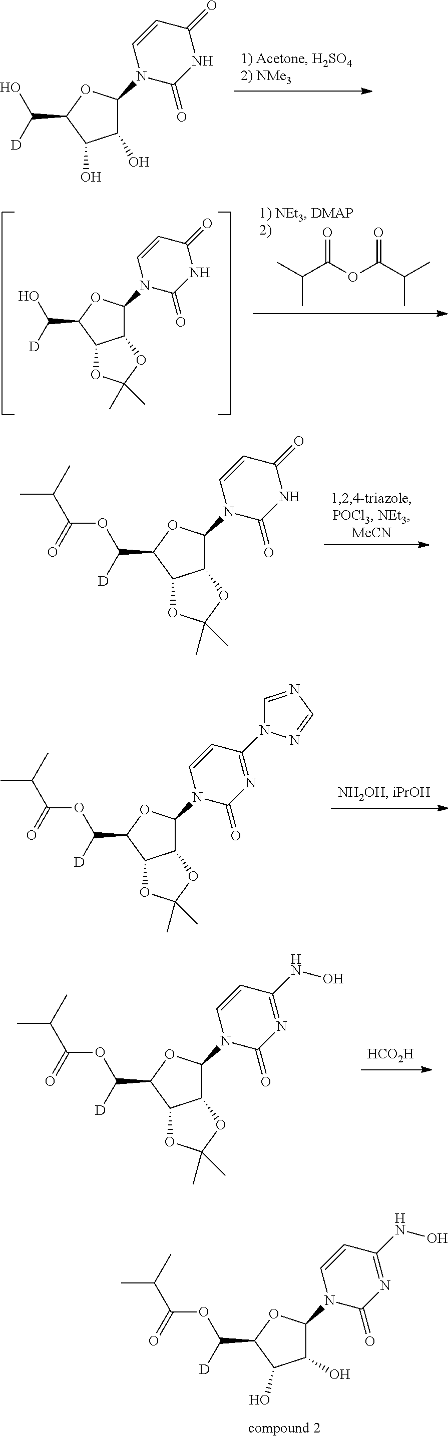

Specific operational steps can be found in CN202111479537.7, the contents of which are expressly incorporated herein.

### Example 3: Cytotoxicity Assay

### Test Method

1) Vero-E6 cells were digested and passaged. The cells were adjusted to a density of 1×10⁵ cells/mL with Dulbecco's Modified Eagle Medium (DMEM) containing 10% fetal bovine serum (FBS), then seeded into a 96-well plate (100 μl/well), and incubated in an incubator at 35° C. and 6% CO₂ for 20 h;
2) The culture medium in the wells was removed. The wells were washed twice with 1×PBS (phosphate-buffered saline) and centrifugal dried. DMEM containing 1% FBS and a composition containing Compound 1 and Compound 2 (mol./mol.=1/1) were then added so that the final combined total concentrations of the compositions containing Compound 1 and Compound 2 in the wells (Compound 1+Compound 2) were 1000 μM, 300 μM, 100 μM, 30 μM, 10 μM, 3 μM, 1 μM, 0.3 μM, 0.1 μM, 0.03 μM, 0.01 μM with a same gradient dilution, and the 96-well plate, including a control without the compounds, was incubated in a 35° C., 6% CO₂ incubator;
3) After 48 h, the cell viability was tested with a CCK8 kit.

### Test Results

The results are shown in FIG. 1. As can be seen from the Figure, the composition had little toxic effect on Vero-E6 cells, and its CC50 was greater than 100 μM.

### Example 4: In Vitro Antiviral Activity EC₅₀ Assay

### Test Method

1) Vero-E6 cells were digested and passaged. The cells were adjusted to a density of 1×10⁵ cells/mL with DMEM containing 10% FBS, then seeded in a 96-well plate (100 μl/well), and incubated in a 35° C., 6% CO₂ incubator for 20 h;
2) Compound 1 and Compound 2 (mol./mol.=1/1) were diluted with DMEM containing 1% FBS to provide compositions with combined total concentrations (Compound 1+Compound 2) of 200 μM, 60 μM, 20 μM, 6 μM, 2 μM, 0.6 μM, 0.2 μM, 0.06 μM, and 0.02 μM;
3) The culture medium in each well was removed, and the 96-well plate was marked on its lid, washed three times with 1×PBS, and 100 μL of dilutions of the compositions prepared above was added to each well, followed by 100 μL of a 0.01 multiplicity of infection (MOI) virus solution, so as to obtain final combined total concentrations of the compounds (Compound 1+Compound 2) at 100 μM, 30 μM, 10 μM, 3 μM, 1 μM, 0.3 μM, 0.1 μM, 0.03 μM, 0.01 μM. The 96-well plate, including a cell control group and a virus control group, was then incubated in a 35° C., 6% CO₂ incubator;
4) After 48 h, the cell viability was tested with a CCK8 kit.

### Test Results

The results are presented in Table 1. The half maximal effect concentration (EC₅₀) of the composition of the present invention was 0.241 μM, significantly lower than that of Compound 1 (1.716 μM) and Compound 2 (1.563 μM) alone.

TABLE 1
Comparison of In Vitro Antiviral Activity
(EC₅₀) of Drug Combinations

| Group | EC₅₀ (μM) |
|---|---|
| Compound 2 | 1.716 ± 0.585 μM |
| Compound 1 | 1.563 ± 0.576 μM |
| Compound 1 + Compound 2 | 0.241 ± 0.084 μM |

### Example 5: Determination of Antiviral Activity (TCID50)

### Test Method

1) Vero-E6 cells were digested and passaged. The cells were adjusted to a density of 1×10⁵ cells/mL with cell culture medium, then seeded into a 96-well plate (100 μL/well), and incubated in a 35° C., 6% CO₂ incubator for 20 h;
2) Compound 1 and Compound 2 (mol./mol.=1/1) were diluted with DMEM containing 1% FBS so as to provide compositions having combined total concentrations (Compound 1+Compound 2) of 200 μM, 60 μM, 20 μM, 6 μM, 2 μM, 0.6 μM, 0.2 μM, 0.06 μM, and 0.02 μM;
3) After removing the culture medium in the wells, the 96-well plate was washed three times with 1×PBS, and 100 μL of dilutions of the compositions prepared above was added to each well, followed by 100 μL of 0.01 multiplicity of infection (MOI) virus per well, so as to obtain final combined total concentrations of the compounds (Compound 1+Compound 2) at 100 μM, 30 μM, 10 μM, 3 μM, 1 μM, 0.3 μM, 0.1 μM, 0.03 μM, and 0.01 μM. The 96-well plate was then incubated in a 35° C., 6% CO₂ incubator;
4) After about 80 h, the supernatant of each well was collected to determine the viral titers.

### Test Results

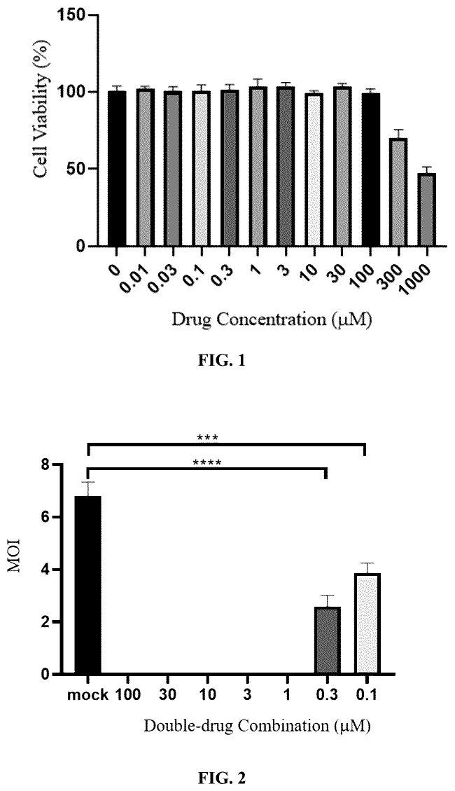

As can be seen from FIG. 2, the viral titer decreases with an increase in the concentration of the composition containing the compounds. The inhibitory effect was most pronounced when the concentration of the composition was greater than 0.3 μM. The TCID50 of the composition was 0, 0, 0, 0, 0, 10^{-2.58}/mL, 10^{-3.87}/mL, and 10^{-6.8}/mL at combined total concentrations (Compound 1+Compound 2) of 100 μM, 30 μM, 10 μM, 3 μM, 1 μM, 0.3 μM, 0.1 μM, and 0 μM, respectively. No virus was detected at 100 μM, 30 μM, 10 μM, 3 μM, and 1 μM and the proliferation of the virus could be completely inhibited. Proliferation of the virus could be decreased by about 4.22 titers at 0.3 μM and about 2.93 titers at 0.1 μM.

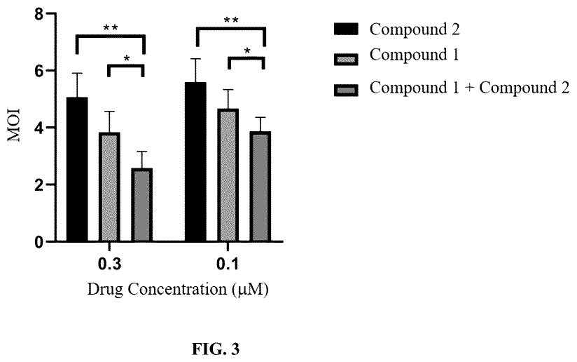

The results of the comparison of the composition containing both Compound 1 and Compound 2 with that of each drug alone are shown in FIG. 3. The TCID50 of 0.3 μM Compound 2 or Compound 1 was 10^{-5.07}/mL and 10^{-3.84}/mL, respectively, while the TCID50 of the 0.3 μM composition containing both Compound 1 and Compound 2 was 10^{-2.58}/mL, which was a decrease of 2.49 titers compared to Compound 1 alone and a decrease of 1.26 titers compared to Compound 2 alone. The TCID50 of 0.1 μM Compound 2 or Compound 1 was 10^{-5.596}/mL and 10^{-4.67}/mL, respectively, and the TCID50 of the 0.1 μM composition containing both Compound 1 and Compound 2 was 10^{-3.87}/mL, which was a decrease of 3.016 titers compared to Compound 1 alone and a decrease of 2.09 titers compared to Compound 2 alone.

In summary, the antiviral effect of the compositions of the invention containing both Compound 1 and Compound 2 is significantly superior to that of either compound alone because of a significant synergistic effect. In addition, the deuterated cyano compound targets viral proteases, whereas the deuterated cytidine derivative targets viral RNA polymerase. Combining drugs that interact with different targets reduces drug resistance and thus improves drug safety.

The above-described embodiments express only several embodiments of the invention, which are described in more detail, but should not be understood as limiting the scope of the invention. It should be noted that for those of ordinary skill in the art, a number of modifications and improvements may also be made without departing from the inventive concept, all of which fall within the scope of protection of the invention.

What is claimed is:

1-10. (cancelled)

11. A pharmaceutical composition comprising a deuterated cyano compound of structure: 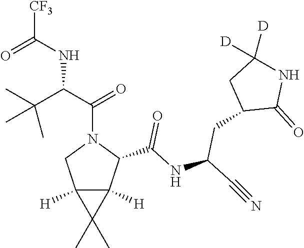 [SMILES: [2H]C1([2H])C[C@@H](C[C@@H](C#N)NC(=O)[C@@H]2[C@@H]3[C@H](CN2C(=O)[C@@H](NC(=O)C(F)(F)F)C(C)(C)C)C3(C)C)C(=O)N1]
or isomer, prodrug, solvate or pharmaceutically acceptable salt thereof, and a deuterated cytidine compound of structure: 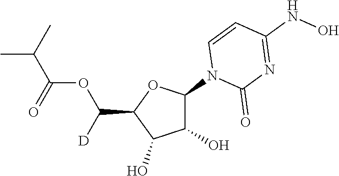 [SMILES: [2H]C(OC(=O)C(C)C)[C@H]1O[C@@H](n2ccc(NO)nc2=O)[C@H](O)[C@@H]1O]
or isomer, prodrug, solvate, or pharmaceutically acceptable salt thereof.

12. The pharmaceutical composition of claim 11, wherein the weight ratio of the deuterated cyano compound, isomer, prodrug, solvate, or pharmaceutically acceptable salt thereof, to the deuterated cytidine compound, isomer, prodrug, solvate, or pharmaceutically acceptable salt thereof, ranges from about 300 mg:200 mg to about 100 mg:400 mg.

13. The pharmaceutical composition of claim 12, wherein the weight ratio of a the deuterated cyano compound, isomer, prodrug, solvate, or pharmaceutically acceptable salt thereof, to the deuterated cytidine compound, isomer, prodrug, solvate, or pharmaceutically acceptable salt thereof is about 200 mg:300 mg.

14. The pharmaceutical composition of claim 11, further comprising one or more pharmaceutically acceptable carriers.

15. The pharmaceutical composition of claim 14, wherein the pharmaceutically acceptable carriers is selected from the group consisting of modifiers, adhesives, lubricants, disintegrants, fillers, colorants, plasticizers, film coating materials, organic solvents, solubilizers, flavoring agents, and combinations thereof.

16. A method of treating a viral infection in a patient comprising administer the patient a deuterated cyano compound of structure: 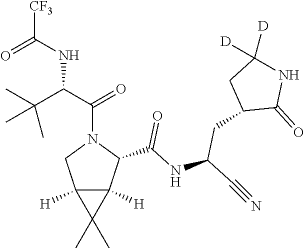 [SMILES: [2H]C1([2H])C[C@@H](C[C@@H](C#N)NC(=O)[C@@H]2[C@@H]3[C@H](CN2C(=O)[C@@H](NC(=O)C(F)(F)F)C(C)(C)C)C3(C)C)C(=O)N1]
or isomer, prodrug, solvate or pharmaceutically acceptable salt thereof, and a deuterated cytidine compound of structure: 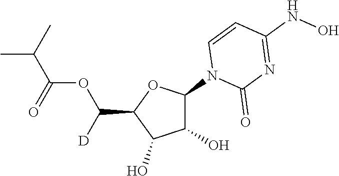 [SMILES: [2H]C(OC(=O)C(C)C)[C@H]1O[C@@H](n2ccc(NO)nc2=O)[C@H](O)[C@@H]1O]
or isomer, prodrug, solvate, or pharmaceutically acceptable salt thereof.

17. The method of claim 16, wherein the deuterated cyano compound, isomer, prodrug, solvate, or pharmaceutically acceptable salt thereof, and the deuterated cytidine compound, isomer, prodrug, solvate, or pharmaceutically acceptable salt thereof, are combined together to form a single dosing unit that is administered to the patient.

18. The method of claim 16, wherein the deuterated cyano compound, isomer, prodrug, solvate, or pharmaceutically acceptable salt thereof, is formulated as a first dosing unit; the deuterated cytidine compound, isomer, prodrug, solvate, or pharmaceutically acceptable salt thereof, is formulated as a second dosing unit; and the first dosing unit and the second dosing unit are separately administered to the patient.

19. The method of claim 16, wherein the viral infection is selected from the group consisting of novel coronavirus SARS-COV-2, SARS-coronavirus, MERS-coronavirus, influenza A virus, influenza B virus, eastern equine encephalitis virus, western equine encephalitis virus, Venezuelan equine encephalitis virus, chikungunya fever virus, orthomyxoviridae virus, paramyxoviridae virus, RSV virus, and Ebola virus.

20. The method of claim 19, wherein the viral infection is selected from the group consisting of the novel coronavirus SARS-COV-2, SARS-coronavirus, MERS-coronavirus, influenza A virus, and influenza B virus.

21. The method of claim 20, wherein the viral infection is the novel coronavirus SARS-COV-2.

22. The method of claim 17, wherein the single dosing unit comprises one or more pharmaceutically acceptable carriers.

23. The method of claim 18, wherein the first dosing unit and the second dosing unit each comprise one or more pharmaceutically acceptable carriers.
